(19) United States
(12) Patent Application Publication
(10) Pub. No.: US 2011/0262521 A1
(43) Pub. Date: Oct. 27, 2011

(54) MEDICAL DEVICE INCLUDING BACTERIAL CELLULOSE REINFORCED BY RESORBABLE OR NON RESORBABLE REINFORCING MATERIALS

(75) Inventors: Yves Bayon, Lyon (FR); Sébastien Ladet, Lyon (FR); Olivier Lefranc, Chatillon sur Chalaronne (FR); Philippe Gravagna, Irigny (FR)

(73) Assignee: SOFRADIM PRODUCTION, Trevoux (FR)

(21) Appl. No.: 13/125,601

(22) PCT Filed: Nov. 6, 2009
(86) PCT No.: PCT/IB2009/007680

§ 371 Date: Jul. 11, 2011

Related U.S. Application Data
(60) Provisional application No. 61/112,302, filed on Nov. 7, 2008.

(51) Int. Cl. A61K 9/70; A61P 17/02; C12P 19/04; A61K 31/717

(52) U.S. Cl. 424/443; 514/57; 435/101

(57) ABSTRACT

The present invention relates to a medical device comprising: a reinforcing member; and bacterial cellulose on at least a portion of the reinforcing member. The invention also relates to a method for making such a medical device.

Medical devices in accordance with this disclosure include bacterial cellulose mechanically reinforced by bioresorbable or non bioresorbable materials. The present disclosure also relates to the use of the medical device for indications where the mechanical constraints are particularly high, for example, in the replacement of tendons and ligaments (referenced hereafter as “orthopedic soft tissues”), and when a permanent reinforcing member is preferred, for example, in the repair of a large hernia. In the present application, the terms “medical device” and “medical implant” have the same meaning. The present disclosure relates to a medical device comprising:
a reinforcing member; and
bacterial cellulose on at least a portion of the reinforcing member.
In embodiments, the reinforcing member comprises a bioresorbable material. In embodiments, the reinforcing member comprises a non-bioresorbable material.
In embodiments, the bacterial cellulose is derived from Acetobacter xylinum.
The bacterial cellulose may be oxidized.
In embodiments, the reinforcing member is a textile made from multifilament yarns, monofilament yarns, or combinations thereof. The reinforcing member may be a non woven structure. The reinforcing member may be a combination of textiles, sheets and fibers.
The present disclosure also relates to a method for making a medical implant comprising:
culturing cellulose-producing bacteria in the presence of at least one reinforcing member thereby forming bacterial cellulose on at least a portion of the reinforcing member.
In embodiments, the bacteria is cultured in a culture vessel. The reinforcing member may be suspended at a distance of about 1 mm to about 3 mm above the bottom of the culture vessel during said culturing. Alternatively, the reinforcing member is disposed at the bottom of the culture vessel during said culturing.
Alternatively, the reinforcing member may fixed at a distance of about 1 mm to about 3 mm above an adhesion surface of the bacteria during said culturing.

The present disclosure also relates to a method of treating a wound comprising contacting a wound with a medical device as described above.

In the present disclosure, the term “bioresorbable” is intended to mean the characteristic according to which an implant and/or a material is resorbed by the biological tissues and the surrounding fluids and disappears in vivo after a given period of time, that may vary, for example, from one day to several months, depending on the chemical nature of the implant and/or of the material.

In the present disclosure, “non bioresorbable” material, also referred to as permanent material, is intended to mean the characteristic according to which an implant and/or material is not substantially resorbed by biological tissues and surrounding fluids and does not disappear in vivo after a given period of time, that may vary, for example, till after 2 years or more, keeping most, for example, at least >80% of their mechanical properties after such a time.

In the present disclosure, the microbial cellulose may be produced as wet pellicles or films from bacteria that synthesize cellulose. Cellulose is synthesized by bacteria belonging to the genera Acetobacter, Rhizobium, Agrobacterium, and Sarcina. Cellulose may be produced by certain bacteria from glucose in the presence of oxygen, (such as, for example, Acetobacter xylinum, referenced hereinafter as the “bacteria”), in static conditions or in a bioreactor (see, e.g. U.S. Pat. Nos. 4,912,049 and 5,955,326, the entire disclosures of which are incorporated herein by this reference). Cellulose suitable for use in the present implants may be obtained by the fermentation of the bacteria. In embodiments, a derivative of the cellulose is employed, such as oxidized cellulose resulting from the oxidation of the cellulose by periodic acid or nitrogen dioxide.

Microbial cellulose possesses inherent characteristics which allow effective promotion of wound healing (see, e.g. U.S. Pat. No. 7,390,492, the entire disclosure of which is incorporated herein by this reference). In this regard, microbial cellulose displays properties (such as a multi-layer three dimensional laminar structure) that distinguish it from plant cellulose and other natural polymeric materials. In this regard, microbial cellulose shows excellent wet strength, does not easily breakdown under compression and demonstrates high moisture handling ability.

In embodiments, cellulose pellicles or films are produced by culturing bacteria in culture vessels or bioreactors in the presence of reinforcing member. As the bacteria grow and proliferate, they form an extracellular network of cellulose on at least a portion of the reinforcing member. The reinforcing member may be any bioresorbable or non bioresorbable material compatible with the culture conditions, allowing the growth of the bacteria and giving a desired mechanical strength to the device of the present disclosure after its final processing.

The reinforcing member may be made from bioresorbable materials, non-bioresorbable materials or combinations thereof.

Suitable examples of non resorbable materials, may include but are not limited to, for example, polyesters, polyolefins, acrylics, fluorocarbons, hydrogels, polyacetals, polyamides, polycarbonates, polyaryletherketone polyimides, polystyrenes, polysulfones, polyurethanes, poly(vinyl chloride), silicone rubbers, polyethylenes, polyetherketones, and combinations thereof.

Suitable examples of bioresorbable materials may include but are not limited to, poly(lactic acid) (PLA), oxidized cellulose, polycaprolactone (PCL), polydioxanone (PDO), trimethylene carbonate (TMC), polyvinyl alcohol (PVA), polyhydroxyalkanoates (PHAs), polyamides, polyethers, copolymers thereof and combinations thereof. The bioresorbable materials may also be obtained from derivatives of polysaccharides among hyaluronic acid, alginic acid, poly(glucuronic acid), chitosan, chitin, cellulose, cross-linked derivatives thereof, and combinations thereof.

The bioresorbable and non bioresorbable materials may be selected from their ability to stand the culture conditions, in an aqueous medium, at a temperature of from about 10° C. to about 40° C., during several days, at a mild acid pH in a range of about 2 to about 6. The bioresorbable and non bioresorbable materials may be selected from their ability to stand the full product processing, including the depyrogenation step as described in, for example, US Patent Publication No. 2007/0128243, the entire content of which is hereby incorporated by reference. The process may be optimized by lowering the temperature and the sodium hydroxide concentration.

In embodiments, the reinforcing member may be a textile which is formed using known techniques such as weaving, braiding or knitting. The textile may be made from multifilament yarns, monofilament yarns, or combinations thereof. The textile may be a two dimensional textile or a three dimensional textile, for example including for the latter spacers, giving controllable thickness to the textile. The yarns may be made from bioresorbable materials, non bioresorbable materials, or combinations thereof such as those identified above.

In embodiments, the reinforcing member may be a sheet, having a structure that is open enough to let the bacteria migrate through the sheet and encapsulate at least a portion of the sheet with the produced cellulose. The sheet may include pores, open to at least at one side of the sheet. In embodiments, the size of the pores may be from about 1 μm to about 10 mm, in embodiments, from about 50 μm to about 5 mm, in other embodiments, from about 500 μm to about 3 mm. In embodiments, the size of the pores may include two or more of these ranges. The softness of the sheet should be in the same order of the tissues to be replaced, such as for example, orthopaedic tissue or abdominal wall tissue. The sheet may be made from bioresorbable reinforcing materials, non bioresorbable materials, or combinations thereof, such as those identified above.

The reinforcing members may be of any shape.

The reinforcing members may have a thickness ranging from about 0.1 mm to 30 mm, in embodiments from about 0.5 mm to 5 mm.

In other embodiments, more than one reinforcing member or more than one type of reinforcing member may be incorporated into the present device.

The bacteria may be grown in the presence of the reinforcing member, either in static culture conditions or in a bioreactor as described in U.S. Pat. Nos. 4,912,049 or 5,955,326. In static culture conditions of the bacteria, the reinforcing member may be laid at the bottom of the culture vessels or at a distance of about 1 mm to about 3 mm above the bottom of the vessels, fixed in position by any appropriate means. The reinforcing member may be fixed in such a way as to give a desired shape to reinforcing member and thereby imparting the desired shape to the cellulose pellicles or films. For example, in embodiments where the reinforcing members are textiles, they may be fixed with enough tension to impart a substantially flat orientation that is parallel to the bottom of the vessels.

When the bacteria are grown in bioreactors, the reinforcing members may be fixed at the surface where the bacteria will adhere and proliferate or at a distance of about 1 mm to about 3 mm above the adhesion surface of the bacteria. The reinforcing members may be fixed in the bioreactor as described hereinabove. For example, in the case of rotating disk bioreactor, the reinforcing members may be laid and fixed on the disks or fixed at a distance of about 1 mm to about 3 mm above the disks. The fixation of the reinforcing members should withstand the shear stress induced by the bioreactor at work on the discs.

In embodiments, a non woven reinforcing member may be formed from fibers of bioresorbable materials, non bioresorbable materials, and combinations thereof, may be embedded in a cellulose pellicle or film obtained by the culture of bacteria. In embodiments, the fibers may be mixed with the culture medium of the bacteria at a predetermined density to allow the formation of the non woven reinforcing member. Suitable densities include but are not limited to from about 0.1 to about 100 grams of fiber per liter of culture medium, in embodiments from about 1 to about 20 grams of fiber per liter of culture medium. In embodiments, the fibers may be added in the culture medium, several hours to a couple of days, after the seeding of the bacteria, in static culture conditions or in bioreactors.

In other embodiments, the bacteria may be grown on a combination, of reinforcing members, including textiles, sheets and fibers.

The replacement or repair of orthopaedic soft tissues may be achieved by the medical device of the present disclosure, which provides sufficient mechanical strength for such repairs.

The implant device may have a final thickness from about 1 mm to about 20 mm, in embodiments, from about 2 mm to about 10 mm.

In embodiments, the final device may be asymmetric in its physical properties. Thus, the device according to the present disclosure may have a mechanical strength, at least in one direction, measured according to ISO standard 13934-1 (properties of substances in tensile testing), from about 250 N to about 1000 N, in embodiments, from about 250 N to about 500 N.

The medical device of the present disclosure may have an elongation from about 1% to about 10% measured at 100 N in the more mechanically resistant direction, measured according to ISO standard 13934-1.

In embodiments, the present reinforced cellulose pellicles or films may be kept wet with a solution, dispersion cellulose concentration or suspension having from about 1% to about 20%. In other embodiments, the cellulose pellicles or films are dried by freeze-drying or by using solvents like water miscible solvents.

In embodiments, the present reinforced cellulose pellicles or films may be compressed using known techniques so as to provide the desired final dimensions and cellulose density.

In embodiments, the present reinforced cellulose pellicles or films are cross-linked. In embodiments, chemical functionalization of the cellulose, provides a desired degradation profile and other features of the final device.

The medical implants in accordance with this disclosure may be produced at a predetermined size or produced in large sheets which may be cut to sizes appropriate for the envisaged application. The medical implants may be packaged in single or dual pouches and sterilized using conventional techniques, such as, but not limited to, irradiation with beta (electronic irradiation) or gamma (irradiation using radioactive cobalt) rays at about 25 KGy to about 35 KGy, and/or sterilized by ethylene oxide. In embodiments where hydrolytically unstable materials are used in forming the implant, such as polyglycolic acid or polylactic acid, the composites are packaged under sufficiently dry conditions to ensure that no degradation of the composite takes place during storage.

The present medical devices including bacterial cellulose mechanically reinforced by bioresorbable or non bioresorbable materials obtained by fermentation of Acetobacter xylinum, may advantageously maintain one or more of the unique properties of bacterial cellulose (such as, for example, high biocompatibility, extreme hydrophilicity, unique multi-layered three dimensional laminar structures which provide its moisture handling properties, excellent wet strength, high resistance to breakdown under compression, conformability, absence of generation of harmful particles of the cellulose mesh after rubbing against surrounding tissues or erosion at sharp edges of tissues—e.g. sharp edges of bone and cartilage tissues) with minimal processing, in particular to keep the shape of the pellicle or film.

The present medical devices may be easily fixed into its desired final shape with the reinforcing member in one step, avoiding the inclusion of reinforcing member by handling the cellulose, and potentially by modifying its unique properties as obtained after the fermentation process.

The medical devices of the present disclosure may be used for challenging surgical conditions where the mechanical constraints are particularly high such as the repair and/or replacement of orthopedic soft tissues.

The medical devices of the present disclosure having non bioresorbable reinforcing members may be used for surgeries requiring the repair of the abdominal wall where the reinforcing member should last more than about 2 to about 3 years.

It will be understood that various modifications may be made to the embodiments disclosed herein. Thus, those skilled in the art will envision other modifications within the scope and spirit of the disclosure.

1-13. (canceled)

14. A medical device comprising: a reinforcing member; and bacterial cellulose on at least a portion of the reinforcing member.

15. The medical device of claim 14, wherein the reinforcing member comprises a bioresorbable material.

16. The medical device of claim 14, wherein the reinforcing member comprises a non-bioresorbable material.

17. The medical device of claim 14, wherein the bacterial cellulose is derived from Acetobacter xylinum.

18. The medical device of claim 14, wherein the bacterial cellulose is oxidized.

19. The medical device of claim 14, wherein the reinforcing member comprises a textile made from multifilament yarns, monofilament yarns, or combinations thereof.

20. The medical device of claim 14, wherein the reinforcing member comprises a non woven structure.

21. The medical device of claim 14, wherein the reinforcing member comprises a combination of textiles, sheets and fibers.

22. A method for making a medical implant comprising: culturing cellulose-producing bacteria in the presence of at least one reinforcing member thereby forming bacterial cellulose on at least a portion of the reinforcing member.

23. The method of claim 22, wherein the bacteria is cultured in a culture vessel.

24. The method of claim 22, wherein the reinforcing member is suspended at a distance of about 1 mm to about 3 mm above the bottom of the culture vessel during said culturing.

25. The method of claim 22, wherein the reinforcing member is disposed at the bottom of the culture vessel during said culturing.

26. The method of claim 22, wherein the reinforcing member is fixed at a distance of about 1 mm to about 3 mm above an adhesion surface of the bacteria during said culturing.
